(19) United States
(12) Patent Application Publication
(10) Pub. No.: US 2008/0312637 A1
(43) Pub. Date: Dec. 18, 2008

(54) Device and Methods for Introducing a Catheter into an Intervertebral Disc

(75) Inventors: David Miller, Cupertino, CA (US); Michael Smith, San Jose, CA (US)

Correspondence Address: HAYNES AND BOONE, LLP, 901 Main Street, Suite 3100, Dallas, TX, 75202, US

(73) Assignee: Kyphon Inc., Sunnyvale, CA (US)

(21) Appl. No.: 11/764,029

(22) Filed: Jun. 15, 2007

(51) Int. Cl. A61M 31/00; A61M 25/06; A61M 25/10

(52) U.S. Cl. 604/512; 604/164.01; 604/164.13; 604/96.01

(57) ABSTRACT

A system includes components that allow the user to select how to perform disc diagnosis with the components of the system. The system components include a FAD catheter, a guidewire and stylet, in which both the guidewire and stylet are compatible with the catheter. The user may select to insert the catheter into the disc with a stylet included in the system. The user may also select to insert the catheter into the disc with the guidewire included in the system, for example after the guidewire has been placed with a rigid needle.

### CROSS-REFERENCES TO RELATED APPLICATIONS

The subject matter of the present application is related to but does not claim the benefit of the following commonly assigned and concurrently filed U.S. patent application Ser. No. ______, filed on Jun. 15, 2007, entitled “SYSTEMS AND METHODS FOR NEEDLE ACCESS TO INTERVERTEBRAL DISC” (Attorney docket no. 019433-002400US); Ser. No. ______, filed on Jun. 15, 2007, entitled “SYSTEMS AND METHODS FOR NEEDLE ACCESS TO INTERVERTEBRAL DISC” (Attorney docket no. 019433-002410US); and Ser. No. ______, filed on Jun. 15, 2007, entitled “SYSTEMS AND METHODS FOR NEEDLE ACCESS TO INTERVERTEBRAL DISC” (Attorney docket no. 019433-002420US), the full disclosures of which are hereby incorporated by reference.

### BACKGROUND OF THE INVENTION

The present invention generally relates to medical devices and methods. More particularly, the present invention relates to devices and methods for diagnosing and/or treating spinal pain.

Back pain takes an enormous toll on the health and productivity of people around the world. According to the American Academy of Orthopedic Surgeons, approximately 80 percent of Americans will experience back pain at some time in their life. In just the year 2000, approximately 26 million visits were made to physicians' offices due to back problems in the United States.

In at least some instances, surgical prostheses can be used to relieve back pain. Many of these prosthesis relieve pressure and/or irritation of nerve roots near the vertebral joints. Such treatments can be effective when the source of pain can be localized to a specific inter-vertebral joint and/or disc.

Unfortunately, back pain can be difficult and invasive to accurately diagnose in an effective manner that determines where the pain originates. Axial pain can be caused by disc failure that results in compression of nerves. “Discogenic pain,” for example, is a type of spinal pain originating in one or more intervertebral discs (soft tissue structures between vertebrae of the spine). The physical examination and complaints of the patient may merely provide general clues as to the cause and general location of the pain.

One approach to determine the source of back pain, can be to perform provocative discography. This procedure can include penetrating the patient's skin and injecting a contrast agent into the disc, and imaging of the disc with fluoroscopy, radiographs, CT scans, or the like with the aid of the contrast agent. Alternatively, a new technique referred to as Functional Anesthetic Discography (FAD) has been developed wherein the disc can be further evaluated with functional tests in which the patient assumes a painful position and the disc is injected with analgesic or similar substance to determine if the pain diminishes in response to the injection into the disc. In some instances, provocative discography may use two needles for each disc. Placement of the needles may require skill and time on the clinicians' part, and can be painful to the patient even when good technique is used. As the patient may have more that one disc that may be a possible source of back pain, each of these suspected discs may be tested to determine which, if any, of the discs is the source of the patient's pain. The testing of multiple discs, for example three discs, can be time consuming and result in multiple injections for the patient, each of which can be painful.

Access to the discs along the spine of the patient can vary, and the clinician may use different methodologies in response to patient variability. Discs higher in the spine, for example L3-L4, may be relatively easy to access with a stylet and catheter, whereas placement at lower levels, such as L5-S1, can be more difficult. With these challenging levels, clinicians may prefer access with standard spinal needles. In some instances, a clinician may not know which approach will be used until after the procedure has started.

For these reasons, it would be desirable to provide apparatus and methods for facilitating the diagnoses and treatment of spinal pain. It would be particularly desirable if such methods and apparatus were less invasive than current methods and apparatus, and assured reliable determination of the source of patient pain, ideally while allowing the clinician flexibility with respect to which approach is used in a manner that accommodates patient variability.

Related devices and procedures are disclosed in U.S. Patent Publication No. 2005/0234425 which describes a Functional Anesthetic Discography (FAD) catheter, the full disclosure of which is incorporated herein by reference. Additional art that may be relevant includes U.S. application Ser. Nos. 11/021,786 and 60/826,472, the full disclosures of which are incorporated herein by reference, and International Publication No. WO 2005/102440.

### BRIEF SUMMARY OF THE INVENTION

Embodiments of the present invention provide flexibility with many of the usage situations described above, can facilitate evaluation of spinal discs, and in at least some instances may permit less invasive FAD and other forms of discography. In many embodiments, system, typically in the form of a kit, is provided which allows the user to select how to perform the disc diagnosis with the components of the system. The system components may include a FAD catheter, a guidewire and stylet, in which both the guidewire and stylet are compatible with the catheter. The user may select to insert the catheter into the disc with a stylet included in the system. As the catheter has a lumen sized to receive either the stylet or the guidewire, the user may select to insert the catheter into the disc with a guidewire included in the system, for example after the guidewire has been placed with a rigid needle. Some embodiments of the present invention can utilize needles that have already been placed to introduce the FAD catheter quickly and safely, so as to avoid additional needle penetration and associated pain. For example, a clinician may access the disc with a standard needle and use this needle to place the catheter, even if a standard discography is not performed.

In a first aspect, embodiments of the present invention provide a system for accessing an interior of an intervertebral disc. The system comprises an intervertebral disc access catheter having at least one lumen for introducing one or more substances into the interior of the intervertebral disc. The system also comprises a stylet that is removably receivable in the at least one lumen. The stylet has a tissue-penetrating distal tip, which extends from a distal end of the catheter when the stylet is in place in the lumen. The tip will usually comprise an integrated sharpened end, but could alternatively comprise a radiofrequency electrode (capable of applying cutting current), a removably or separately formed sharpened end, or the like. A removable guidewire that is receivable in the at least one lumen is also included in the system. A user can select to advance percutaneously the access catheter to the disc interior using the stylet, or the user can select to remove the stylet and introduce the access catheter over a guidewire to the disc interior.

In many embodiments, the intervertebral disc access catheter comprises an elongate flexible catheter body. The catheter body may have a proximal portion and a distal portion. The disc access catheter may comprise an inflatable anchoring balloon on the distal portion of the catheter body and an injection tube or lumen in the elongate body. The injection tube or lumen may be adapted to removably receive either the guidewire or the stylet. In specific embodiments, the intervertebral disc catheter may comprise an inflation tube or lumen in the elongate body.

In many embodiments, the intervertebral disc access catheter has an outer diameter in the range from 0.4 mm to 1.5 mm, and an injection lumen diameter in the range from 0.08 mm to 0.8 mm. The disc access catheter may have an inflation lumen diameter in the range from 0.08 mm to 0.8 mm, and a length in the range from 10 cm to 62 cm. The guidewire may have a diameter in the range from 0.02 mm to 0.8 mm, and the stylet may have a diameter in the range from 0.02 mm to 0.8 mm and a length in the range from 20 cm to 80 cm. The guidewire may has a length in a range from about 20 cm to about 80 cm.

In another aspect, embodiments of the present invention provide a method for accessing the interior of an intervertebral disc. The method may comprise providing a system as described above, and the access catheter may be advanced to the disc interior using the stylet.

In many embodiments, a method for accessing the interior of an intervertebral disc is provided. The method may comprise providing a system as described above. A needle may be percutaneously advanced into the interior of the intervertebral disc, and the catheter may be advanced over the guidewire so that a distal end of the catheter is positioned in the disc.

In many embodiments, a method for assessing the condition of an intervertebral disc may be provided. The method may comprise percutaneously advancing a needle into an intervertebral disc. The condition of the disc may be assessed using X-ray. If further assessment is desired, a guidewire may be advanced through the needle in response to the condition assessed with X-ray, and the needle may be removed over the guidewire in response to the condition assessed with X-ray. A catheter may be advanced over the guidewire in response to the condition assessed with X-ray, so that a distal end of the catheter is positioned in the disc. At least one anesthetic or analgesic may be introduced through the catheter. The condition of the disc may be further assessed based on the patient's response to the anesthetic or analgesic.

In many embodiments, a method for assessing the condition of an intervertebral disc having an annulus and a nucleus is provided. A system is provided which comprises a needle, a catheter, a stylet and a guidewire. The stylet is provided inside a lumen of the catheter. Either the guidewire or the stylet is selected to position a distal end of the catheter through the annulus into the nucleus. The distal end of the catheter is advanced into the nucleus with either the guidewire or the stylet in response to the selection.

In specific embodiments, the needle can be selected in response to a location of the disc in the patient, and the needle can be selected in response to a previously placed catheter.

In some embodiments, the system is comprised within a sterile package comprising a kit, and a plurality of sterile kits are provided to the physician for assessing a plurality of discs. In specific embodiments, each kit of the plurality of kits corresponds to an evaluated disc of the plurality of discs.

### BRIEF DESCRIPTION OF THE DRAWINGS

FIG. 1A shows a kit, according to embodiments of the present invention;

FIGS. 1B and 1C show components of the kit as in FIG. 1A in detail, according to embodiments of the present invention;

FIGS. 2A-2D show a method of accessing the interior of an intervertebral disc with components of a kit as in FIG. 1A, in which an access catheter can be advanced into the interior of the disc with a stylet, according to embodiments of the present invention; and

FIGS. 3A-3K show a method of accessing the interior of an intervertebral disc with components of a kit as in FIG. 1A, in which a needle can be percutaneously advanced to the interior of the disc and a catheter can be advanced over a guidewire positioned with the needle, according to embodiments of the present invention.

### DETAILED DESCRIPTION OF THE INVENTION

Embodiments of the present invention can be used in many flexible ways to benefit clinical procedures, including Functional Anesthetic Discography (FAD) and traditional provocative discography (PD). For example, a clinician may use kit components to place a FAD catheter at all disc levels being tested and perform a provocative discography with each placed FAD catheter. Alternatively or in combination, the clinician may use the kit components to perform a traditional provocative discography and place a FAD catheter at the level of the disc in response to the disc testing is positive. The FAD catheter anchor may be deployed and the functional portion of the FAD testing conducted in response to the positive test result of the disc. In some embodiments, the clinician may place a FAD catheter from the kit without performing the provocative discography, for example in response to physical exams, imaging studies such as x-ray or MRI, or previous discographies that have been performed.

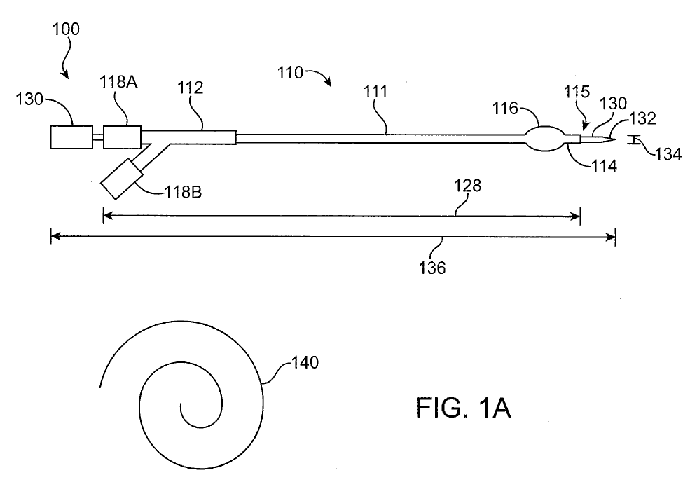

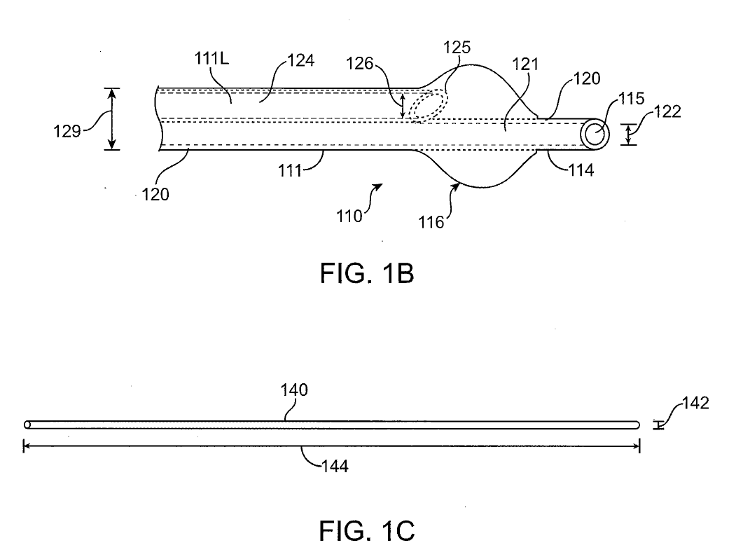

Referring now to FIG. 1A, a schematic illustration of a system, or kit 100, is shown, according to embodiments of the present invention. Kit 100 comprises a catheter 110, a stylet 130 and a guidewire 140. Catheter 110 may comprise an elongate and flexible catheter body 111. Catheter body 111 comprises a proximal portion 112 and a distal portion 114. An adapter 118A and an adapted 118B may be connected to catheter 110 near proximal portion 112. Catheter 110 comprises a length 128. Length 128 may comprise many suitable lengths, and length 128 may comprise a range of lengths from about 10 to 62 cm. Stylet 130 passes though an opening 115 formed near the distal end of catheter 110. Catheter 110 may comprise many suitable materials, for example braid reinforced polyimide tubing, and many materials as described in U.S. application Ser. No. 10/825,961, the full disclosure of which has been previously incorporated by reference.

An anchor 116 is located near distal portion 114 of the catheter body. Anchor 116 may comprise an expandable member, such as an expandable balloon, to anchor the catheter in the disc. Anchor 116 may comprise many suitable structures that anchor the catheter in and/or near the disc. In many embodiments, anchor 116 may comprise an expandable balloon that can be inflated upon injection of a fluid through inflation tube 125. Many suitable anchor structures are described in U.S. application Ser. No. 10/825,961, the full disclosure of which has been previously incorporated by reference. For example, the expandable anchor may comprise flexible polyvinyl chloride (PVC), Polyethylene, Polyether Block Amide (PEBAX), Polyethylene Terepthalate (PET), Polyester, Nylon, Polyurethanes, Polyether Block Amide (PEBAX), Polyolefins or any suitable combination thereof. Various adhesives may be used to attach anchor 116 to the catheter shaft or for any other suitable purpose. Many suitable adhesive(s) may be used, such as but not limited to, light activated acrylics, light activated cyanoacrylates, light activated silicones, heat activated adhesives, ambient curing adhesives, cyanoacrylates, epoxy adhesives, and/or polyurethane adhesives. Various parts of the catheter device may also be attached using alternative means, such as friction fitting, snap fitting, screw fitting, application of energy such as thermal or radiofrequency energy, and/or the like.

Stylet 130 can be sized to pass through catheter 110 and may comprise a sharpened distal tip 132 adapted to penetrate tissue and permit catheter body 111 to enter tissue. Stylet 130 comprises a cross-sectional size, for example a diameter 134, to permit stylet 130 to slide through connector 118A and catheter body 111. Outside diameter 134 of stylet 130 may comprise a range of diameters from about 25% to 75% of the overall outside diameter of catheter 110, for example from about 0.005″ to about 0.035″. Stylet 130 comprises a length 136. Length 136 may comprise many suitable lengths, and length 136 may comprise a range of lengths from about 20 to 80 cm. In many embodiments, stylet 136 may stiffen catheter 110 to facilitate placement. Stylet 130 may comprise many suitable materials such as stainless steel, tungsten, tungsten alloys, cobalt chrome alloys, nitinol, molybdenum rhenium alloys, Eligloy, MP35N, or L605, 35NLT. Additionally the stylet could be treated with materials to enhance its radiopacity such as gold, platinum, iridium, or alloys thereof or materials to enhance it lubricity such as Teflon, PTFE, or parylene.

Guidewire 140 can be sized to pass through catheter 110. In many embodiments, catheter body 111 can be advanced over guidewire 140 when stylet 130 has been removed from catheter 110. Guidewire 140 may be advanced into the disc and catheter 110 advanced over the guidewire to permit anchor 116 to attach to the disc. Guidewire 140 may comprise a cross-sectional size, for example a diameter 142, and a length 144 when elongate, as shown in FIG. 1C. Diameter 142 may comprise a range from about 0.02 mm to about 0.8 mm. Length 144 may comprise a range from about 20 to 80 cm. In an exemplary embodiment, catheter 110 may comprise an overall outside diameter of about 0.038″ with an injection lumen of about 0.018″ that can accommodate a 0.017″ guidewire and a 0.017″ stylet. Guidewire 140 may comprise many suitable materials such as stainless steel, tungsten, tungsten alloys, cobalt chrome alloys, nitinol, molybdenum rhenium alloys, Eligloy, MP35N, or L605, 35NLT. Additionally the guidewire could be treated with materials to enhance its radiopacity such as gold, platinum, iridium, or alloys thereof or materials to enhance it lubricity such as Teflon, PTFE, or parylene.

Referring now to FIG. 1B, catheter 110 is shown in detail near distal end portion 114, according to embodiments of the present invention. Catheter body 111 comprises an inner lumen 111L with an injection tube 120 disposed therein that extends from attachment 118A to distal portion 114. Injection tube 120 comprises a lumen 121 with a size, for example, an inner diameter 122, to permit stylet 130 to slide through tube 120. Injection tube 120 extends from attachment 118A to opening 115 such that fluids can be injected from attachment 118A to the disc through injection tube 120. Inner diameter 122 may comprise a range of diameters, for example a range from about 0.08 to 0.8 mm. Catheter body 111 may also comprise within lumen 111L an inflation tube 125, which comprises an inflation lumen 124. Inflation tube 125 and inflation lumen 124 extend from attachment 118B to anchor 116. Inflation lumen 124 comprises an inner size, for example a diameter 126. Diameter 126 may comprise a range from about 0.08 to 0.8 mm. Catheter 110 may comprise an overall outside diameter 129 in a range from about 0.38 mm (0.015″) to about 1.5 mm (0.060″).

Catheter injection lumen 121 may be sized to receive guidewire 140 and/or stylet 130. Such sizing of the tube lumen, stylet and guidewire provide flexible use of the kit components. For example, the user may select to insert catheter 110 with stylet 130. Guidewire 140 may also be used to insert catheter 110. The user may remove stylet 130 such that injection lumen 120 is available, and the user may insert guidewire 140 into lumen 121 and advance catheter 110 into the patient, with guidewire 140 inserted into injection lumen 120 so as to guide the catheter to the disc.

Kit 100 can be shipped to the clinician in many configurations. Kit 100 can be shipped to the clinician with stylet 130 positioned within catheter 110. In some embodiments, the kit can be shipped to the clinician with guidewire 140 and stylet 130 removed from catheter 110 and ready for placement within the catheter.

Provocative discography may be used with at least some embodiments of the present invention. Provocative discography may provide anatomical and functional information about the evaluated disc. Provocative discography may comprise an imaging procedure in which a contrast agent may be injected into the nucleus pulposus. Following injection, the disc can be evaluated on radiographs and/or computed tomography (CT).

Some embodiments of the present invention may use functional evaluation of the disc. Functional evaluation may comprise pain provocation and careful assessment of the patient's response to pain. A substance can be injected into the nucleus pulposus that may reduce pain perceived by the patient. For example, if the patient reports a decrease in pain after injection of the substance into the disc, the disc may contribute to pain previously perceived by the patient and the defective disc may be corrected surgically.

Several substances may be injected into the nucleus pulposus to perform the functional evaluation. In some embodiments, at least one of the following substances can be introduced: an anesthetic; an analgesic; an antibiotic; a hydrating agent such as hypotonic saline, isotonic saline or hypertonic saline; a supportive agent such as a hydrogel, ethylene-vinyl alcohol copolymer, Dimethyl Sulfoxide or Tantalum; a prolotherapy agent such as sodium morrhuate, cod oil, phenol, minerals or ethyl alcohol; and other agents such as collagen, stem cells, Osteogenic Protein-1, ethanol, alcohol, steroids, radio-opaque contrast agents, ultrasound contrast agent, Bone Morphogenetic Protein (BMP), BMP-2, BMP-4, BMP-6, BMP-7, BMP-12, Serotonin 5-HT2A receptor inhibitors, LMP-1, TIMP-1, TGF-1, TGF-2, Rofecoxib, Ketorolac, Glucosamine, Chondroitin Sulfate, Dextrose, DMSO, non-steroidal antiinflammatory drugs, ibuprofen, naprosyn, Bextra, Vioxx, Celebrex, indomethacin, botulinum toxin, capsaicin, vanilloid agonists, vanilloid antagonists, VR1, VRL-1, steroids, methylprednisolone or chymopapain; cells, cell fragments, tissue, tissue gragments; genetic material, such as DNA, cDNA, RNA, mRNA, rRNA, siRNA, tRNA, plasmids, lentivirus, adenovirus, adeno-associated virus, or derivatives or fragments or synthetic mimics thereof, cytokines, growth factors, differentiation factors, hormones, ligands, receptors; intracellular regulatory molecules, or transcription factors, or their agonists, antagonists, activators, or inhibitors, or derivatives or fragments of synthetic mimics thereof, matrix molecules such as fibrin, collagen, proteoglycans, glycosaminoglycans, polysaccharides, elastin, or derivatives or fragments or synthetic mimics thereof, matrix-regulating molecules such as crosslinking agents, link protein, metalloproteinases, or enzymes, or their activators or inhibitors, or derivatives or fragments or synthetic mimics thereof, drugs such as statins, purmorphamine, anti-inflammatory drugs; neurotransmitting agents or neurotoxic agents or their inhibitors; MRI contrast agents; bone fillers, bone graft materials, and bone graft substitutes such as bone autograft, bone allograft, anorganic bone matrix, demineralized bone matrix, calcium phosphate, tricalcium phosphate, calcium sulfate, hydroxyapatite, bioglass, polymers, or combinations thereof, additional biologic-based, biologic derived, or biologic-mimicking substances; and substances used for controlled release of any of the above substances, such as polymers, liposomes, self-assembling monolayers, tie-layer molecules, scaffolds, or gels.

In some embodiments, the substance can be injected to alter the pH of the nucleus. In specific embodiments directed to diagnosis, raising the pH can make the nucleus and surrounding tissues more basic so as to lower the threshold of triggering nociceptive receptors. Such pH lowering substances can also be injected into the patient in therapeutic embodiments.

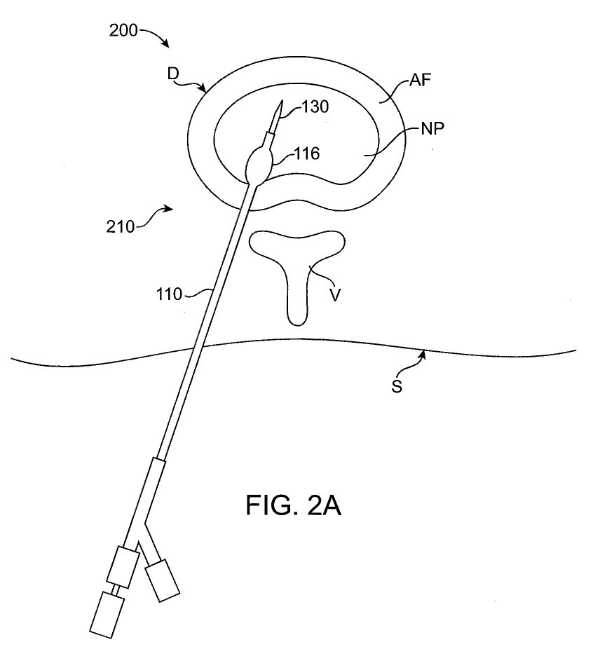

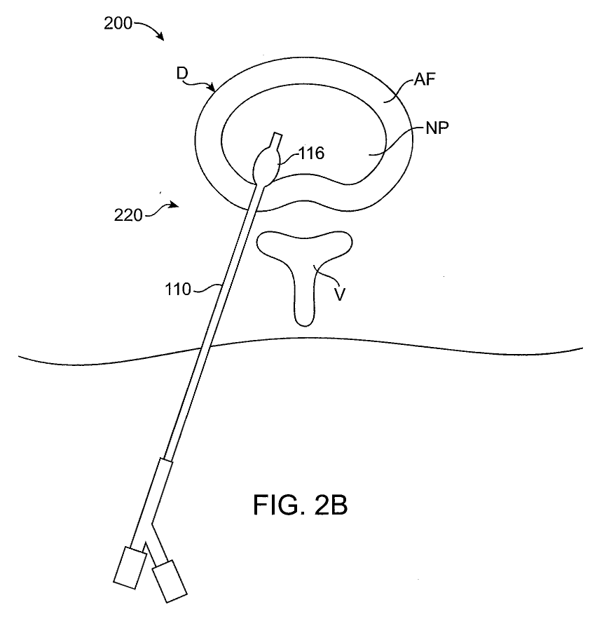

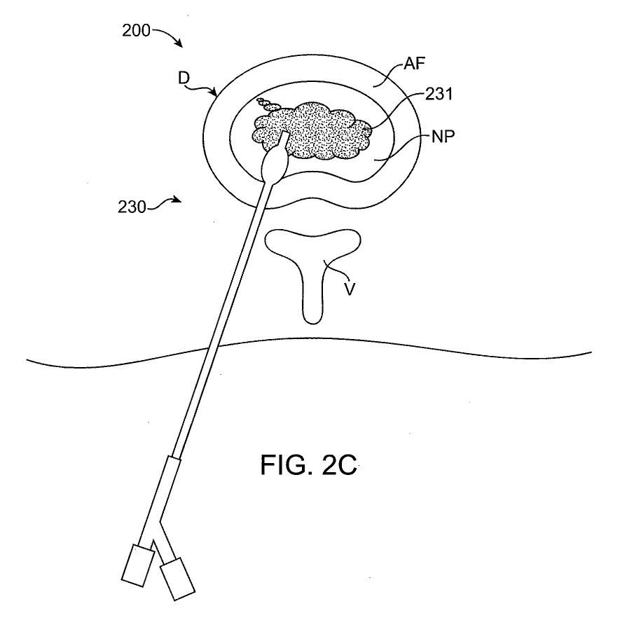

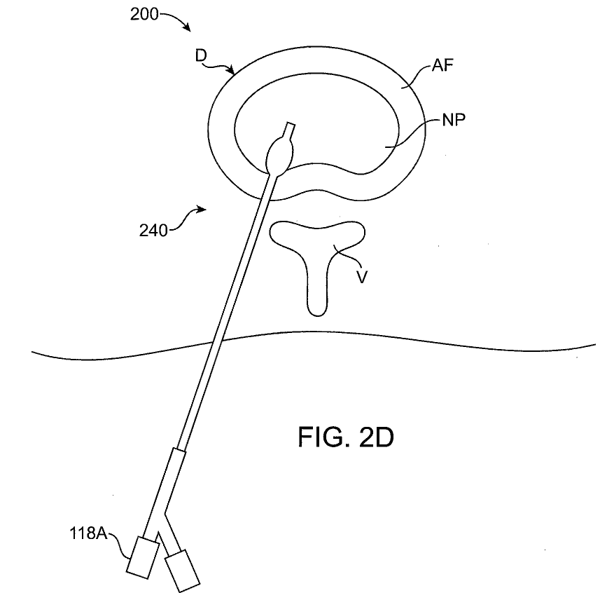

FIGS. 2A to 2D show a method 200 of accessing the interior of an intervertebral disc with an access catheter advanced into the interior of the disc with a stylet, according to embodiments of the present invention. A step 210 delivers catheter 110 with a stiffened stylet 130 from a kit as described above. A skin S is penetrated by stylet 130 and catheter 110, and the stylet and catheter are advanced distally past a vertebra V into a disc D. Disc D comprises an annulus fibrosis AF, or annulus, and a nucleus pulposus NP, or nucleus. After anchor 116 of catheter 110 has been introduced into disc D, anchor 116 is expanded to anchor catheter 110 in disc D. In some embodiments, stylet 130 penetrates the annulus fibrosis to enter the nucleus pulposus and anchor 116 is deployed at least partially within the nucleus pulposus. A step 220 may remove the stylet and may deploy anchor 116 to anchor catheter 110 with anchor 116. A step 230 may perform a provocative discography PD to assess disc D. In some embodiments, a traditional PD may be performed and a contrast agent 231 can be injected at adapter 118A to pass through the injection lumen into the disc. A step 240 may perform a functional test. The patient may be asked to assume a position that induces pain, and a substance such as an anesthetic and/or analgesic or the like introduced, and the patient asked if the pain has decreased to determine if the disc is the source of pain experienced by the patient.

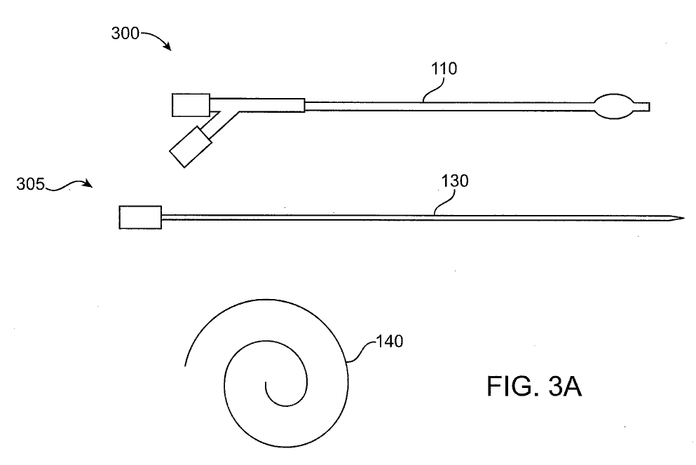

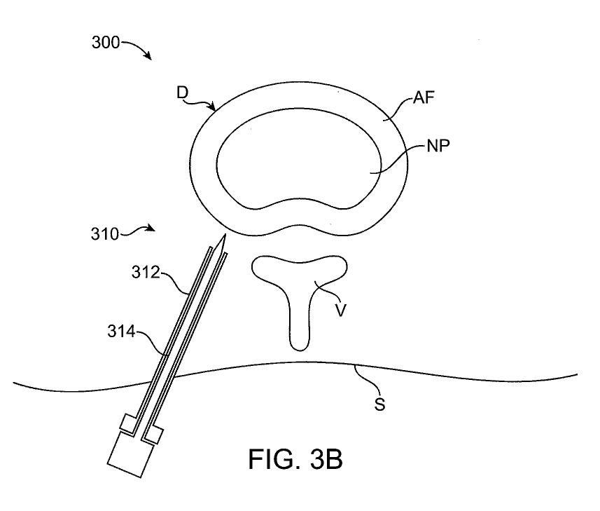

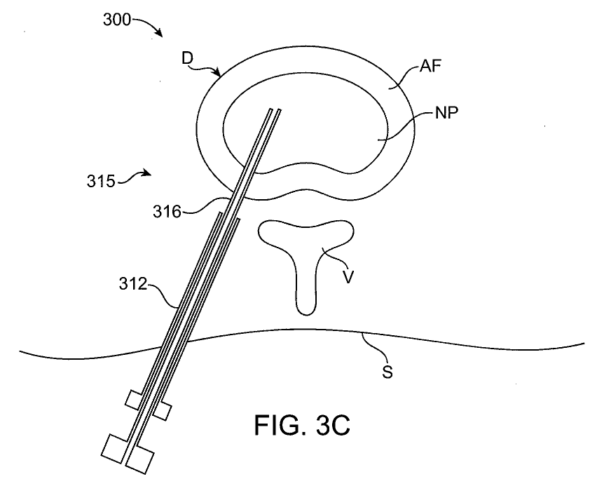

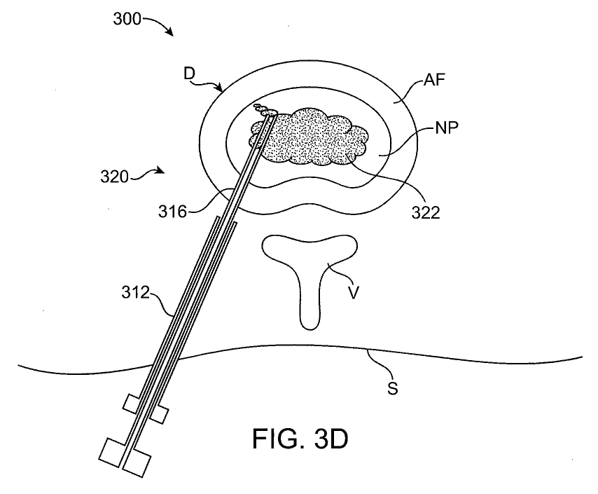

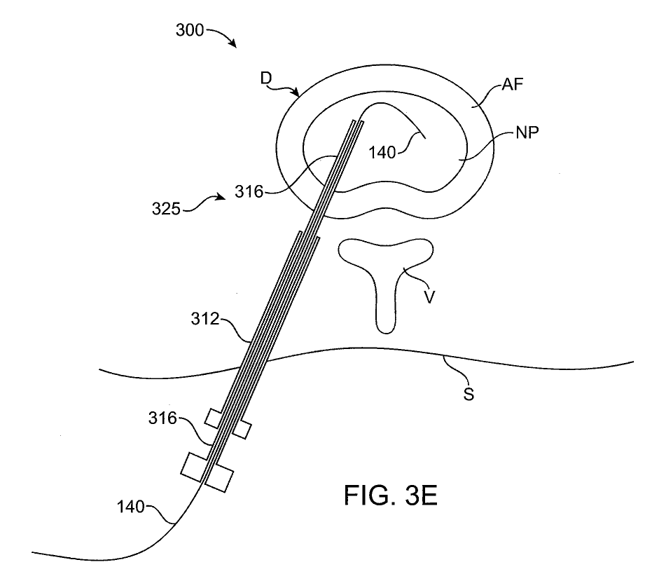

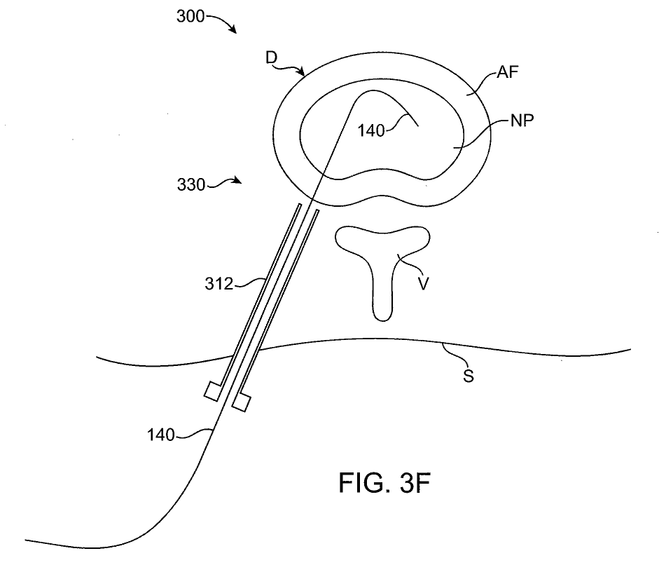

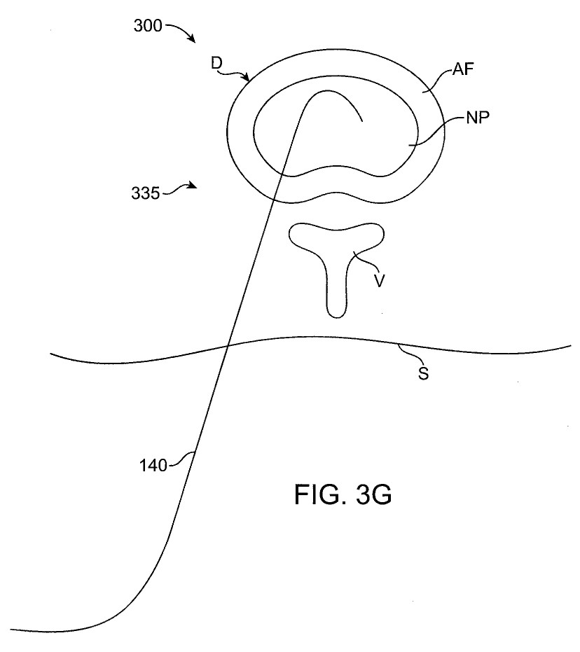

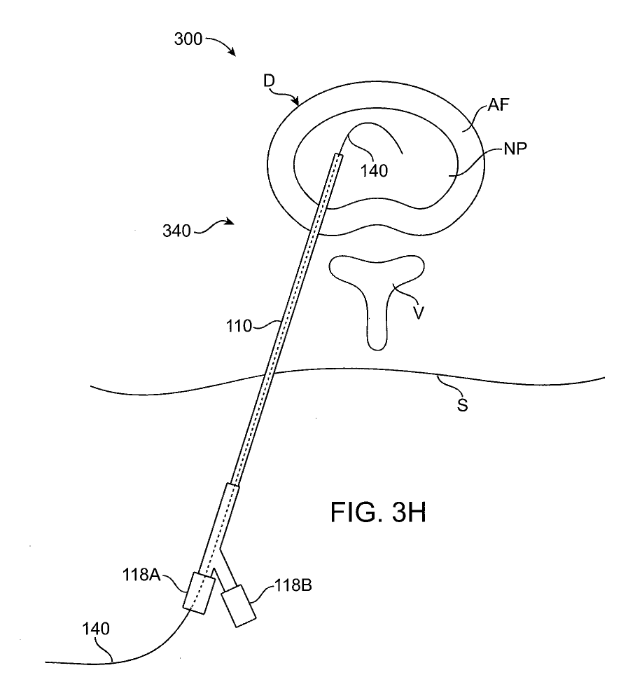

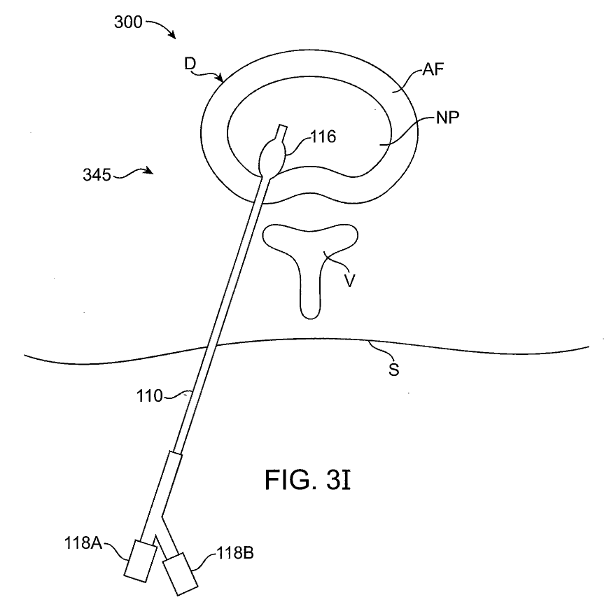

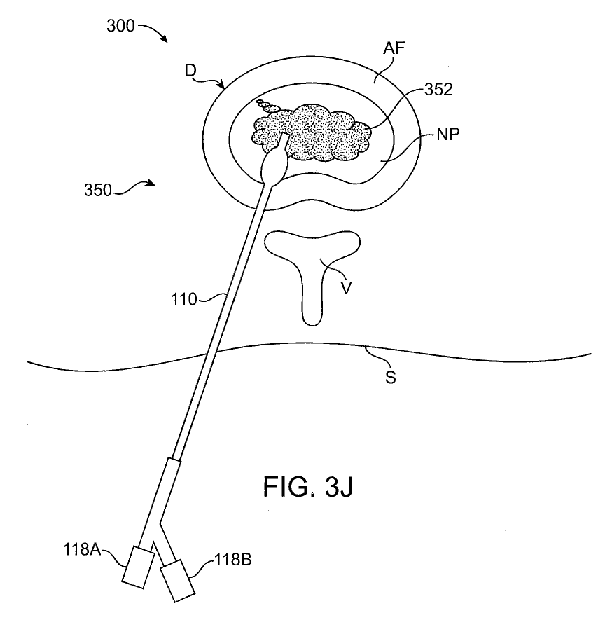

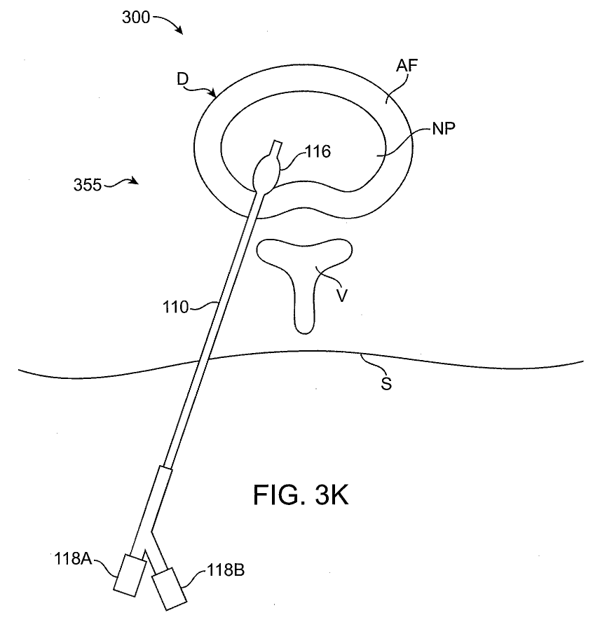

FIGS. 3A to 3K show a method 300 of accessing the interior of an intervertebral disc with a needle percutaneously advanced to the interior of the disc, and a catheter advanced over a guidewire positioned with the needle, according to embodiments of the present invention. A step 305 removes stylet 130 from catheter 110 of the kit as described above. Guidewire 140 remains separate from catheter 110. A step 310 advances an introducer, or outer needle 312, and a stylet 314 through skin S of the patient. A step 315 removes stylet 314 and passes an inner needle 316 into the disc space within disc D. Inner needle 316 may penetrate the annulus fibrosis and enter the nucleus pulposus. A step 320 may perform a provocative discography, for example a traditional discography. A contrast agent 322 that can be detected by fluoroscopy and/or X-rays, MRI or the like can be injected into disc D. A step 325 may pass guidewire 140 from a proximal end of inner needle 316, through the inner needle, out a distal end of inner needle 316 and into the nucleus pulposus. A step 330 may remove inner needle 316 while guidewire 140 remains positioned in the nucleus pulposus and outer needle 312 remains positioned through skin S. A step 335 may remove outer needle 312 and guidewire 140 remains position through skin S and extends into the nucleus pulposus. A step 340 may deliver catheter 110 with the guidewire through skin S, the annulus fibrosis and into the nucleus pulposus. Catheter 110 can slide over guidewire 140 with guidewire 140 extending through adapter 118A. In some embodiments, adapter 118A may be removed while the catheter is advanced over the guidewire and attached after the catheter is placed in the disc. A step 345 removes guidewire 140 and deploys anchor 116. A step 350 may perform a provocative discography, for example if a traditional discography with a needle as described above has not been performed previously. A contrast agent 352 can be injected through catheter 110 and the disc D imaged, for example with fluoroscopy. A step 355 may perform a functional test that includes an injected substance as described above. For example, an anesthetic and/or analgesic substance may be injected through catheter 110 while anchor 116 is deployed, and patient response evaluated as described above.

It should be appreciated that the specific steps illustrated in FIGS. 3A to 3K provide a particular method of accessing the interior of an intervertebral disc, according to an embodiment of the present invention. Other sequences of steps may also be performed according to alternative embodiments. For example, alternative embodiments of the present invention may perform the steps outlined above in a different order. Moreover, the individual steps illustrated in FIGS. 3A to 3K may include multiple sub-steps that may be performed in various sequences as appropriate to the individual step. Furthermore, additional steps may be added or removed depending on the particular applications. One of ordinary skill in the art would recognize many variations, modifications, and alternatives.

In some embodiments, the needle can be selected in response to a location of the disc in the patient, and the needle can be selected in response to a previously placed catheter. In specific embodiments, the system is comprised within a sterile package comprising a kit, and a plurality of sterile kits are provided to the physician for assessing a plurality of discs. In specific embodiments, each kit of the plurality of kits corresponds to an evaluated disc of the plurality of discs. For example, three sterile kits can be provided to assess three adjacent discs. The decision to use either the needle or the stylet can be based on the catheter previously inserted into the adjacent disc. As the components of each kit are substantially similar, improved consistency in FAD catheter placement can be achieved.

While the above includes a complete description of the preferred embodiments of the present invention, other embodiments may fall within the spirit and scope of the invention. Therefore, the scope of the present invention should be determined with reference to the appended claims along with their full scope of equivalents.

1-9. (canceled)

10. A method for assessing the condition of an intervertebral disc, said method comprising:
percutaneously advancing a needle into an intervertebral disc;
assessing the condition of the disc using X-ray;
advancing a guidewire through the needle in response to the condition assessed with X-ray;
removing the needle over the guidewire in response to the condition assessed with X-ray;
advancing a catheter over the guidewire in response to the condition assessed with X-ray, so that a distal end of the catheter is positioned in the disc;
introducing at least one anesthetic or analgesic through the catheter; and further assessing the condition of the disc based on the patient's response to the anesthetic or analgesic.

11. A method for assessing the condition of an intervertebral disc having an annulus and a nucleus, said method comprising:
providing a system comprising a needle, a catheter, a stylet and a guidewire, wherein the stylet is provided inside a lumen of the catheter;
selecting either the guidewire or the stylet to position a distal end of the catheter through the annulus into the nucleus; and
advancing the distal end of the catheter into the nucleus with either the guidewire or the stylet in response to the selection.

12. A method as in claim 11, wherein the needle is selected in response to a location of the disc in the patient.

13. A method as in claim 11, wherein the needle is selected in response to a previously placed catheter.

14. A method as in claim 11, wherein the system is comprised within a sterile package comprising a kit, and a plurality of sterile kits are provided to the physician for assessing a plurality of discs.

15. A method as in claim 14, wherein each kit of the plurality kits corresponds to an evaluated disc of the plurality of discs.

16. The method of claim 11, comprising:
withdrawing the selected guidwire or the stylet from the catheter while maintaining the catheter in place; and
injecting a contrast agent into the intervertebral disc.

17. The method of claim 11, comprising injecting a fluid material into the nucleus through the catheter.

18. The method of claim 17, wherein injecting a fluid material into the nucleus comprises injecting the fluid material into an inflatable member on the catheter disposed in the nucleus.

19. The method of claim 17, wherein injecting a fluid material into the nucleus comprises releasing the fluid from the catheter into the nucleus.

20. The method claim 19, including imaging the fluid in the nucleus with fluoroscopy.

21. The method of claim 11, wherein the providing step includes providing a system comprising a catheter having a first and a second lumen, the first lumen having an opening contained within an inflatable expandable member.

22. The method of claim 11, including percutaneously advancing a needle into an intervertebral disc.

23. The method of claim 11, including:
assessing the condition of the disc using X-ray;
advancing a guidewire through the needle in response to the condition assessed with X-ray.

24. The method of claim 23, including:
removing the needle over the guidewire in response to the condition assessed with X-ray; and
advancing a catheter over the guidewire in response to the condition assessed with X-ray, so that a distal end of the catheter is positioned in the disc.

25. The method of claim 23, including:
introducing at least one anesthetic or analgesic through the catheter; and
further assessing the condition of the disc based on the patient's response to the anesthetic or analgesic.

26. The method of claim 11, wherein the providing step includes providing a stylet and guidewire having substantially the same sized outer diameter.
